(19) United States
(12) Patent Application Publication
(10) Pub. No.: US 2009/0062922 A1
(43) Pub. Date: Mar. 5, 2009

(54) METHOD AND APPARATUS FOR DELIVERING TREATMENT TO A JOINT

(76) Inventor: WILLIAM F. McKAY, Memphis, TN (US)

Correspondence Address: Medtronic Spinal and Biologics;Attn: Noreen Johnson - IP Legal Department, 2600 Sofamor Danek Drive, Memphis, TN, 38132, US

(21) Appl. No.: 11/850,108

(22) Filed: Sep. 5, 2007

(51) Int. Cl. A61F 2/30; A61F 13/20

(52) U.S. Cl. 623/18.11; 604/11

(57) ABSTRACT

The present invention relates to an apparatus and method for treating inflammation and/or infection within a synovial joint. The invention is comprised of a depot, tether and cap. The depot is comprised of a biodegradable polymer which is impregnated with a biological agent targeted to treat the inflammation and/or infection. The depot is inserted into the synovial joint through an incision or hole in the synovial membrane. A tether that extends from the depot is then thread back through the incision or hole and coupled to a cap such that the depot is coupled to the synovial membrane on an interior side of the synovial joint capsule and the cap is secured to the membrane on an exterior side of the synovial joint capsule. Once secured, the depot degrades and gradually releases the biological agent over a sustained time period. The biological agent then treats the targeted inflammation and/or infection.

### FIELD OF THE INVENTION

The present invention relates to an apparatus and method for treating a disorder or trauma to a joint of a subject's body. More specifically, the present invention relates to surgically implanting a depot within the joint such that the depot delivers a biological agent to the joint. In one embodiment, the depot contains a biological agent that is implanted on the interior surface of the joint capsule of a synovial joint, such as, but not limited, to a knee joint. The depot may be biodegradable such that, as the depot degrades within the body, the biological agent is gradually released to treat the targeted disorder or trauma.

### BACKGROUND OF THE INVENTION

Synovial joints, such as the knee, are joints of the body wherein two adjacent bones are coupled and encapsulated within a synovial membrane or capsule. Referring to FIG. 1A and FIG. 1B, one embodiment of a synovial joint is illustrated as, but not limited to, a human knee joint 1. The joint 1 is comprised of the tibia 5 and the fibula 10 extending up from the lower leg, the femur 15 extending down from the thigh and the patella 40 as the knee cap over the joint. The medial collateral ligament 20 and the lateral collateral ligament 25 connect the femur 15 to the tibia 5 and fibula 10, respectively, and restrict the sideways motion of the joint. The posterior cruciate ligament 30 connects the femur 15 to the tibia 5 and restricts backward movement of the joint away from the patella 40. The anterior cruciate ligament 35 connects the femur 15 to the tibia 5 and restricts the joint rotation and forward motion toward the patella 40. A synovial membrane 45 substantially surrounds the joint and encapsulates the synovial fluid that fills the joint forming the joint capsule. The synovial fluid functions to both lubricate and nourish the joint. Accordingly, the synovial joint functions to facilitate full range of normal articulation and movement of the joint that is unique to each subject. As such, maintaining the integrity the joint is of utmost importance in performance of a subject's day-to-day activities.

There are numerous traumas and/or acute or chronic disorders which affect the normal workings of joint and require therapeutic intervention. Examples of joint disorders include, but are not limited to, osteoarthritis, chondromalacia and rheumatoid arthritis. Examples of joint traumas include, but are not limited to, tearing and/or fracturing of the anterior cruciate ligament, posterior cruciate ligament, the medial collateral ligament, the lateral collateral ligament, the patellar ligament, the medial meniscus, the lateral meniscus and chondrol fractures. Additionally, the joint could simply be infected from a post-surgical or prior joint injury. In each of these disorders and traumas the joint is mechanically compromised either acutely or chronically causing the body to elicit an immune response. Such response is typically manifest in the form of inflammation and/or persistent pain in the joint area.

Inflammation can be an acute response to trauma or a chronic response to the presence of inflammatory agents brought about by any number of processes or events which trigger tissue damage within the synovial joint. For example, when tissues are damaged, tumor necrosis factor-alpha (hereinafter “TNF-a”) attaches to cells causing them to release other cytokines leading to an increase in inflammation. One type of recruited immune system cell is the macrophage. Macrophages release interleukin-1 beta (“IL-β”) and tumor necrosis factor-alpha (“TNF-a”), pro-inflammatory cytokines heavily involved in orchestrating the immediate and local physiological effects of injury or infection. For instance, once released, pro-inflammatory cytokines promote inflammation. The purpose of the inflammatory cascade is to promote healing of the damaged tissue. However, once the tissue is healed the inflammatory process does not necessarily end. Left unchecked, this can lead to degradation of surrounding tissues and associated chronic pain. Thus, pain can become a disease state in itself. That is, when this pathway is activated, inflammation and pain ensue. Cycles of inflammation and associated pain often times set in.

Current treatment methods of inflammation of the joints include the use of biological agents which are designed to reduce inflammation such that the pain associated with the inflammation subsides and the subject regains at least partial use of the joint. Such biological agents include, but are not limited to, analgesics and an anti-inflammatory drugs. These drugs are known to be administered orally and/or may be directly injected into the inflamed joint. However, these types of treatment methods only reduce inflammation for a limited time span. Thus, they are required to be administered regularly by the subject or his/her attending physician. Recently, however, there have been a number of attempts to develop implants that would administer the desired biological agent gradually and continuously over a longer time frame.

One such current method of administering the biological agent is through non-injectable implants such as depots. A depot is a device that contains and gradually releases a biological agent to a targeted region over time. One such example of a depot is a capsule that contains the biological agent within a biocompatible housing wherein the end caps of the capsule are comprised of a biodegradable polymer. A second example of a depot is a biodegradable capsule wherein the biological agent is distributed homogenously throughout the capsule. In either case, as the biodegradable polymer dissolves in the body, the biological agent is gradually released into the synovial capsule.

For example, U.S. Patent Application Publication No. 20050152949 to Hotchkiss et al. and U.S. Patent Application Publication No. 20030139811 to Watson both disclose a drug release device or depot designed for implantation into an inflamed and/or infected synovial joint. According to the disclosure of the Hotchkiss publication, the depot is comprised of a capsule that contains the implantable drug release device that contains a biological agent within the chamber of the capsule. At one end of the capsule is a biodegradable polymer. In use, the depot is implanted into the bone of a knee joint within the synovial capsule such that the polymer end of the depot is exposed to the synovial fluid. As the polymer degrades, the biological agent is gradually released into the synovial fluid to treat the synovial joint. The Watson publication is similar to the Hotchkiss publication however, in Watson, the depot is in the form of a bone screw. Nonetheless, both methods of the above two publications require compromising the integrity of the actual bone within the joint. Specifically, the implant must be installed by drilling a hole into the bone or installing a prosthetic device within the bone to receive the depot. Moreover, in order to achieve uniform distribution of the agent throughout the joint, several holes must be drilled throughout the bone such that numerous depots are installed. The long term effects of this method could lead to a weakening of the bone structure causing a fracture, infection, etc. within the synovial joint.

U.S. Patent Application Publication No. 20060246103 to Ralph et al. discloses a depot that may be implanted within a bone or prosthesis of a synovial joint or inserted into a mesh bag that is attached to a bone fastener or sutured to a soft tissue, such as a ligament. Much like the Hotchkiss publication, the depot in the Ralph publication requires a hole to be drilled into the bone of the joint to implant the depot. This, as stated above, weakens the integrity of the bone and, ultimately, the joint. Moreover, suturing the depot to a ligament requires temporary damage to the ligament which can also disrupt the integrity of the joint and cause further damage and inflammation.

Accordingly, despite current knowledge in the field covering the surgical and non-surgical treatment of traumas or disorders of the joints, there remains a need for an improved method of treating the acute and chronic inflammation and/or pain associated with recovery from, treatment or prevention of these disorders. Specifically, there remains a need for a prolonged treatment apparatus and method that can treat traumas and/or disorders of the synovial joint without successive oral and/or injected administration and without compromising the integrity of the joint structure or causing further damage to the joint.

### SUMMARY OF THE INVENTION

The present invention relates to an apparatus and method for providing treatment within a synovial joint such as, but not limited to, a knee joint. The method involves surgically implanting a pharmaceutical depot containing a biological agent into the membrane of the synovial joint capsule such that treatment may be administered to the joint from the depot without interfering with the normal articulation of the joint. Surgically implanting the depot can also include making a hole in the synovial membrane by spreading apart the synovial membrane using a blunt instrument and inserting the depot through the hole.

The present invention relates to an apparatus and method for implanting a depot onto the synovial membrane of a synovial joint capsule. More specifically, the present invention is comprised of a depot, a tether, and a cap. The depot may be rod or disc shaped, comprised of a biodegradable polymer and contain a biological agent designed to treat a condition within the synovial joint including, without limitation, inflammation and/or infection. Extending from the depot is a tether that may extend from either the center or either end of the depot. The tether may be rod shaped and its distal end may be adapted to snap fit or press fit within a hole in a corresponding cap such that the tether and cap secure the depot to the synovial membrane.

The device is implanted by first surgically making an incision or hole in the synovial membrane of the targeted joint, in this case a knee joint. The depot is then inserted through the incision or hole such that the depot is on the interior side of the joint and the tether extends back through the incision in the membrane. The cap is secured to the distal end of the tether wherein securing the cap to the tether functions to hold the depot in place within the joint capsule while it gradually administers the biological agent to the targeted region. The cap and depot may also function to seal the incision or hole created in the synovial membrane such that neither synovial fluid nor biological agent leaks from the synovial membrane.

In one embodiment, the cap may be adapted to snap-fit to the tether. To this end, the distal end of the tether may be comprised of a single or a plurality of ridges which frictionally secure the tether to a hole or slot within the cap. Alternatively, the ridges and slot may be adapted to threadingly engaged one another.

In an alternative embodiment, the tether may be adapted to receive a rod shaped cap through a hole in the distal end of the tether. The diameter of the cap is approximately the same size as the diameter of the hole in the tether. In operation, rather than snap fitting the cap over the tether as in the first embodiment, the cap is inserted or threaded through the hole in the distal end of the tether to secure the device to the membrane. Optionally, at least one ridge may extend from the cap such that the ridge(s) frictionally secure the cap to the tether.

In a further embodiment, the tether may be at least one suture extending from the depot. In operation, after inserting the depot into the joint capsule, the tether is thread back through the membrane and may be tied to a cap or secured to the cap by a plurality of beads or knots spaced along the tether. By securing the tether to the cap, the depot is held in place within the joint capsule. Moreover, the cap may also function to seal the incision or hole created in the synovial membrane.

The present invention further relates to an apparatus and method of sustained-release of a biological agent within the synovial space of the joint to promote prophylactic or therapeutic indications contemplated herein. As noted above, the depot may be comprised of a biodegradable polymer incorporated or impregnated with a biological agent. In a first embodiment, the polymer or combination of polymers used depend upon the half-life of the polymer and the rate at which degradation of the polymer would release the biological agent. Ideally, a polymer should be selected that would release the biological agent at a rate to maximize the effectiveness of the treatment sought over a pre-selected period.

The biological agent(s) used in the apparatus and methods of the present invention may be any molecule, cell, or physical stimulus which provides therapeutic or prophylactic relief for acute or chronic pain and/or inflammation associated with any synovial trauma or disorder, including, but not limited to, traumas and disorders associated with the knee joint. The biological agent includes, but is not limited to, an anti-inflammatory agent, an antibiotic and/or an analgesic, or combinations thereof. Each of the above biological agents may be presented in a sustained-release formulation as a pharmaceutical depot implant. Such anti-inflammatory agents may be in any form such that administration of the entity promotes the desired anti-inflammatory response, including any molecule, cell or physical stimulus which positively effects the activity of an anti-inflammatory response. As discussed herein, a targeted inflammatory cytokine or protein related to the inflammatory response includes, but is not limited to, TNF-α, IL-1β, IL-6, IL-8, NF-κB, High Mobility Group Box 1 (HMG-B1), IL-2, IL-15, and matrix metalloproteases (MMPs). A specific anti-inflammatory cytokine or related protein which may promote an anti-inflammatory response includes, but is not limited to, IL-10, IL-4, IL-13 and TGF-β, as well as any other cytokine or pathway related protein which modulates the respective anti-inflammatory cytokine so as reduce patient inflammation and pain within the synovial joint. Additionally, the biological agent(s) may be comprised of an antibiotic, a steroidal anti-inflammatory, and/or a non-steriodal anti-inflammatory. Additionally, such agents may include a small molecule, an oligonucleotide, an antibody or relevant fragment, siRNA, as well as any factor in the form of a molecule cell or physical stimulus which regulates expression of a gene of interest or effects stability or activity of the expression and/or translation of a protein, so as to modulate the target so as to provide a level of relief to the knee joint.

Accordingly, the sustained-release of the contemplated biological agent(s) after the implantation of the depot in the synovial membrane will result in local, biologically effective concentrations of the biological agent(s) in or around the inflamed or infected joint over a period of time.

An object of the present invention relates to the implantation of a sustained-delivery device into the synovial membrane of a synovial joint, such as the knee.

An object of the present invention relates to the use of sustained-delivery devices to treat a synovial joint trauma and/or disorder, where parenteral administration of such a device is accomplished while maintaining normal articulation of the joint.

Another object of the present invention relates to the sustained-delivery of a biological agent from an implanted depot to provide an effective and inexpensive method of providing care. This object of the invention includes, but is not limited to, situations whereby a biological agent is delivered to prevent and/or treat the onset of osteoarthritis, rheumatoid arthritis, and chondromalacia, or to provide therapeutic intervention in order to positively modulate osteoarthritis, rheumatoid arthritis, and chondromalacia.

Another object of the present invention is to provide for sustained-delivery of a biological agent from an implanted drug delivery device within a synovial joint to treat a trauma or disorder within such joint such that the use of the device obviates the need for regular dosing by the patient, thus increasing patient compliance with a prescribed therapeutic regimen, or in particular compliance with a prophylactic regimen prescribed prior to the onset of symptoms.

### BRIEF DESCRIPTION OF THE DRAWINGS

FIG. 1A illustrates a rear view of a synovial knee joint.

FIG. 1B illustrates a side view of a synovial knee joint.

FIG. 2A illustrates an exploded side view of a first embodiment of the drug implant device.

FIG. 2B illustrates a top view of a first embodiment of the drug implant device.

FIG. 2C illustrates a side view of a first embodiment of the drug implant device secured to a synovial membrane.

FIG. 3A illustrates an exploded side view of a second embodiment of the drug implant device.

FIG. 3B illustrates a side view of a second embodiment of the drug implant device secured to a synovial membrane.

FIG. 4A illustrates an exploded side view of a third embodiment of the drug implant device.

FIG. 4B illustrates a side view of a third embodiment of the drug implant device secured to a synovial membrane.

FIG. 5A illustrates an exploded side view of a fourth embodiment of the drug implant device.

FIG. 5B illustrates a side view of a fourth embodiment of the drug implant device secured to a synovial membrane.

FIG. 5C illustrates a side view of an alternative fourth embodiment of the drug implant device secured to a synovial membrane wherein the device is comprised of more than one tether.

FIG. 6 illustrates a side view of the first embodiment of the drug implant device secured to the synovial membrane of a synovial knee joint.

FIG. 7A illustrates an exploded side view of an alternative cap/tether of the first embodiment of the drug implant device.

FIG. 7B illustrates a side view of an alternative cap/tether of the first embodiment of the drug implant device wherein the device is secured to a synovial membrane.

FIG. 8A illustrates an exploded side view of another alternative cap/tether of the first embodiment of the drug implant device.

FIG. 8B illustrates a side view of another alternative cap/tether of the first embodiment of the drug implant device wherein the device is secured to a synovial membrane.

FIG. 9A illustrates an exploded side view of an alternative cap/tether embodiment of the third embodiment of the drug implant device.

FIG. 9B illustrates a top view of an alternative cap/tether of the third embodiment of the drug implant device.

FIG. 9C illustrates a side view of an alternative cap/tether embodiment of the third embodiment of the drug implant device wherein the device is secured to a synovial membrane.

FIG. 10A illustrates a first step in implanting the drug implant device by inserting a cannula through a synovial membrane and threading the depot through the cannula.

FIG. 10B illustrates a second step in implanting the drug implant device by retracting the cannula such that the depot remains on the interior of the synovial membrane and the tether crosses the membrane.

FIG. 10C illustrates a third step in passing the cap through the cannula such that it may be contacted with and secured to the tether.

FIG. 10D illustrates a cap secured to the tether such that the depot is secured to the interior of the synovial membrane.

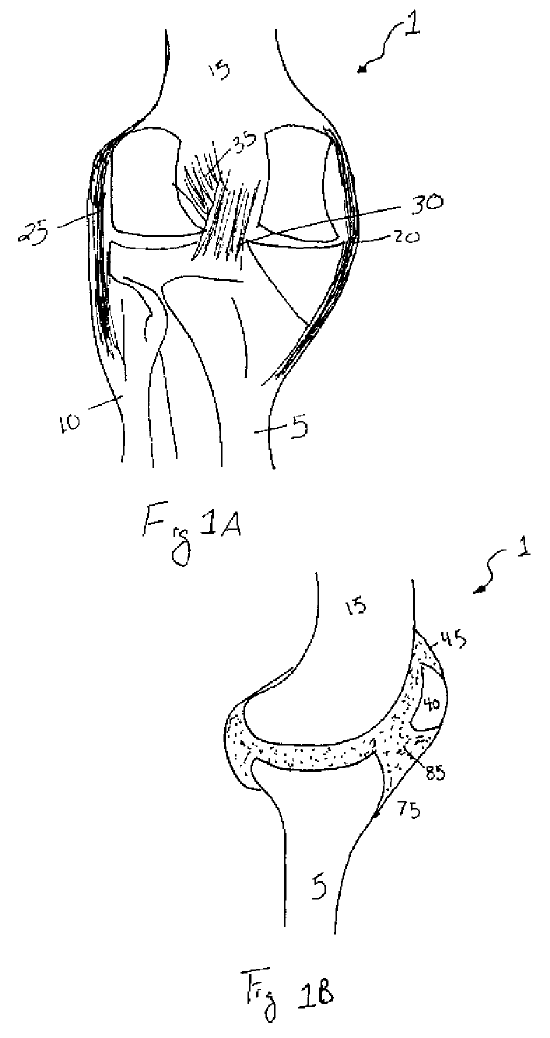

### DETAILED DESCRIPTION OF THE INVENTION

For the purposes of promoting and understanding the principles of the invention, reference will now be made to numerous embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The present invention relates to an apparatus and method for providing treatment within a synovial joint. The treatment comprises administering to the synovial joint space of the subject in need of treatment a pharmaceutically effective amount of a pharmaceutical composition comprising one or more biological agents wherein the biological agents are administered by a sustained-release drug implant. In a particular embodiment of the present invention, the biological agent including, but not limited to, an anti-inflammatory agent, antibiotic, antiviral, analgesic, or other joint therapy agent will be presented in a sustained-release formulation as a depot implant. As discussed throughout this specification, a pharmaceutical depot implant will be inserted within a synovial joint capsule such as, but not limited to, the knee by being secured by a tether and a cap to the capsule membrane. The implantation of such a device will be in such a manner as to allow for normal joint articulation, post-administration, while acting as an adequate reservoir for the prolonged release of the biological agent(s) during the rehabilitation period, wherein normal joint articulation may be defined as, but is not limited to, any range of motion of the joint, as constrained by the ligaments of the joint. The drug delivery device will be capable of carrying the biological agent(s) in such quantities as therapeutically or prophylactically required for treatment over a pre-selected period. The device may also provide protection to the biological agent(s) from premature degradation by body processes (such as proteases) for the duration of treatment. The sustained-release of the contemplated biological agent(s) and any additional active ingredient, carrier or excipient will result in local, biologically effective concentrations of the biological agent(s) in or around an inflamed or infected joint.

Referring to FIG. 2A, FIG. 2B, and FIG. 2C a first embodiment of the present invention is illustrated. In the first embodiment, a sustained-release drug device 51 is illustrated. The sustained-release drug device 51 is comprised of a depot 55, a tether 60, and a cap 70. The depot 55 may be a rod, as illustrated in FIG. 2, a disc, a cylinder, a capsule, a microsphere, a particle, a matric, a wafer, a pill or any other shape understood in the art to act as a sustained-release drug device or depot. The depot 55 may take the form of any solid, biodegradable, natural or synthetic polymer or combinations thereof, as discussed below, and may contain at least one biological agent, as discussed below.

In the first embodiment of the device 51, the tether 60 may be, but is not limited to, a rod-like structure extending from the depot 55 at approximately a perpendicular angle relative to and centered upon the depot 55. The length of the tether 60 may be of any length necessary for implantation into the synovial joint capsule. However, in the first embodiment, the tether is preferably the approximate width of the synovial membrane 45, as illustrated in FIG. 2C, such that the end distal of the tether 65 may be exposed on the exterior side 75 of the synovial membrane 45 and the depot 55 is coupled to the synovial membrane 45 on the interior side 85 of the synovial joint 1. In this first embodiment, the tether 60 is adapted at its distal end to receive a disc-shaped cap 70 with a hole or slot 90 in the center through a snap fit or press fit mechanism. More specifically, the distal end 65 of the tether may be, but is not limited to, a flexible cone shape wherein distal end is slightly larger than the hole 90 of the cap 70. The distal end 65 of the tether 60 may be inserted into the hole 90 of the cap 70 and the cap 70 may be snapped or pressed over the distal end 65 of the tether 60 such that the cap 70 is retained on the tether 60. For example, the cap 70 may be retained by a ridge 71 on the distal end 65 of the tether 60 wherein the circumference of the ridge 71 is slightly larger than the circumference of the hole 90 in the cap. Both the tether 60 and the cap 70 may take the form of a solid, biodegradable, natural or synthetic polymer or any combination thereof, as discussed below, and may be comprised of the same material as the depot 55 or material distinguishable from the depot 55. In one embodiment, the biodegradable polymers for the cap, tether and the depot may be selected to ensure that the depot and the cap will degrade at approximately the same time such that the healing of the incision in the synovial membrane coincides with the degradation of the depot. In an alternative embodiment, both the tether 60 and the cap 70 may take the form of a solid, non-biodegradable, natural or synthetic polymer or any combination thereof. In this embodiment, the tether 60 and the cap 70 do not dissolve and thus ensure that the depot 55 does not become disengaged from the synovial membrane prior to the complete degradation of the depot.

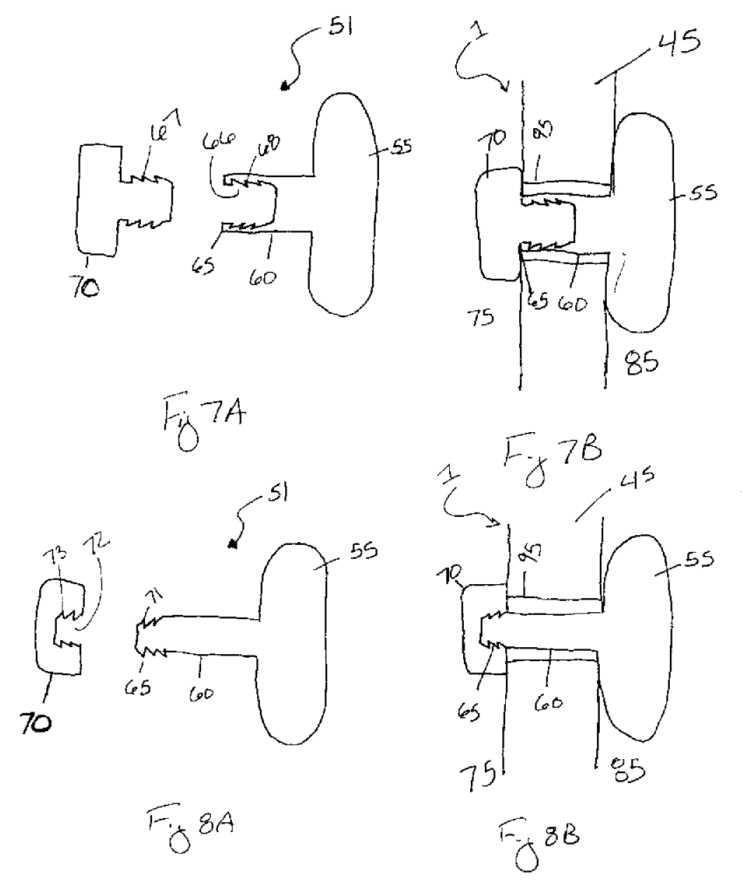

The above first embodiment of present invention is not limited to the recited structure of the depot 55, tether 60, and cap 70. More specifically, as illustrated in FIGS. 7A and 7B, the distal end 65 of the tether 60 may be adapted to receive the cap 70. For example, the tether 60 may contain a slot 66 with at least one groove 68 therein wherein the slot 66 functions as a female end of the tether 60. The cap contains at least one ridge 67 extending from one side of the cap wherein the ridges 67 function as a male end of the cap 70. To this end, the slot 66 is adapted to receive the ridge(s) 67 such that the ridge(s) 67 of the cap 70 are press fit or snapped into the slot 66 of the distal end 65 of the tether 60. Alternatively, the ridge 67 and groove 68 may be adapted such that the cap threadingly engages the tether. In either case, the groove(s) 68 of the slot 66 match up with the ridge(s) 67 of the cap such that the cap 70 is frictionally secured within the tether. As illustrated in FIG. 7B, length of the tether 60 is the approximate width of the synovial membrane 45 such that the tether 65 may be exposed on the exterior side 75 of the synovial membrane 45 and the depot 55 is coupled to the synovial membrane 45 on the interior side 85 of the synovial joint 1.

Alternatively, the male and female ends of the cap and tether may be reversed, as illustrated in FIGS. 8A and 8B. More specifically, at least one ridge 71 may extend from the distal end 65 of the tether 60 wherein the ridge(s) 71 function as the male end of the tether 60. The cap 70 contains a slot 72 with at least one groove 73 therein wherein the slot 72 functions as a female end of the cap 70. To this end, the slot 72 is adapted to receive the ridge(s) 71 such that the ridges 71 of the tether 60 are press fit or snapped into the slot 72 of the cap 70. Alternatively, the ridge 71 and groove 73 may be adapted such that the cap threadingly engages the tether. In either case, the groove(s) 73 of the slot 73 match up with the ridge(s) 71 of the tether such that the tether 60 is frictionally secured within the cap. As illustrated in FIG. 8B, length of the tether 60 is the approximate width of the synovial membrane 45 such that the tether 65 may be exposed on the exterior side 75 of the synovial membrane 45 and the depot 55 is coupled to the synovial membrane 45 on the interior side 85 of the synovial joint 1.

Biodegradable, natural and synthetic polymers known in the art are useful as medical implants due to the versatile degradation kinetics, safety, and biocompatibility of the polymers. These copolymers can be manipulated to modify the pharmacokinetics of a biological agent, shield the agent from enzymatic attack, as well as degrade over time at the site of attachment such that the biological agent is released. It is understood in the art that there are ample teachings to manipulate the properties of these copolymers, including the respective production process, catalysts used, and final molecular weight of the sustained-release depot implant. Natural polymers include, but are not limited to, proteins (e.g., collagen, albumin or gelatin); polysaccharides (cellulose, starch, alginates, chitin, chitosan, cyclodextrin, dextran, hyaluronic acid) and lipids. Biodegradable synthetic polymers may include, but are not limited to, various polyesters, copolymers of L-glutamic acid and gamma ethyl-L-glutamate (Sidman et al., 1983, Biopolymers 22:547-556), polylactides ([PLA]; U.S. Pat. No. 3,773,919 and EP 058,481), polylactate polyglycolate (PLGA) such as polylactide-co-glycolide (see, for example, U.S. Pat. Nos. 4,767,628 and 5,654,008), polyglycolide (PG), polyethylene glycol (PEG) conjugates of poly(α-hydroxy acids), polyorthoesters, polyaspirins, polyphosphagenes, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, polyorthoesters (POE), or any combinations thereof, as described above (see, for example, U.S. Pat. No. 6,991,654 and U.S. Pat. Appl. No. 20050187631, each of which is incorporated herein by reference in its entirety), hydrogels (see, for example, Langer et al., 1981, J. Biomed. Mater. Res. 15:167-277; Langer, 1982, Chem. Tech. 12:98-105, non-degradable ethylene-vinyl acetate (e.g. ethylene vinyl acetate disks and poly(ethylene-co-vinyl acetate)), degradable lactic acid-glycolic acid copolyers such as the Lupron Depot™, poly-D-(-)-3-hydroxybutyric acid (EP 133,988), hyaluronic acid gels (see, for example, U.S. Pat. No. 4,636,524), alginic acid suspensions, polyorthoesters (POE), and the like. Polylactide (PLA) and its copolymers with glycolide (PLGA) have been well known in the art since the commercialization of the Lupron Depot™, approved in 1989 as the first parenteral sustained-release formulation utilizing PLA polymers. Additional examples of products which utilize PLA and PLGA as excipients to achieve sustained-release of the active ingredient include Atridox (PLA; periodontal disease), Nutropin Depot (PLGA; with hGH), and the Trelstar Depot (PLGA; prostate cancer).

Other synthetic polymers include, but are not limited to, poly(c-caprolactone), poly3-hydroxybutyrate, poly(β-malic acid) and poly(dioxanone); polyanhydrides, polyurethane (see WO 2005/013936), polyamides, cyclodestrans, polyorthoesters, n-vinyl alcohol, polyethylene oxide/polyethylene terephthalate or Dacron®, polyphosphazene, polyphosphate, polyphosphonate, polyorthoester, polycyanoacrylate, polyethylenegylcol, polydihydropyran, and polyacytal. Non-biodegradable devices include but are not limited to various cellulose derivatives (carboxymethyl cellulose, cellulose acetate, cellulose acetate propionate, ethyl cellulose, hydroxypropyl methyl cellulose) silicon-based implants (polydimethylsiloxane), acrylic polymers, (polymethacrylate, polymethylmethacrylate, polyhydroxy(ethylmethylacrylate), as well as polyethylene-co-(vinyl acetate), poloxamer, polyvinylpyrrolidone, poloxamine, polypropylene, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-a-chloro-p-xylene, polymethylpentene, polysulfone and other related biostable polymers.

Accordingly, the composition of the depot 55, the tether 60, and the cap 70 of the present invention may include any of the above natural or synthetic, biodegradable or biostable (non-biodegradable) polymers, or combinations thereof. Additionally, with respect to the depot 55, such polymers may be liquid or solid and may be formed into monolithic or coaxially extruded rods, capsules, microspheres, particles, gels, coating, matrices, wafers, pills or other pharmaceutical delivery compositions that may be impregnated or incorporated with a biological agent. In one embodiment, the biodegradable polymers for the cap, tether and the depot are selected to ensure that the depot and the cap will degrade at approximately the same time such that the healing of the incision in the synovial membrane coincides with the degradation of the depot. In an alternative embodiment, the tether and cap are made from biostable polymers while the depot is a biodegradable polymer. In this alternative embodiment, the depot does not dislodge or disengage from the synovial membrane prior to the degradation of the depot because the tether and cap do not degrade. There is no harm to the patient to have a biostable cap and tether.

Various methods for forming a polymer incorporated with a biological agent are known in the art. For example, U.S. Pat. Nos. 5,268,178 and 5,681,873 disclose two such examples and are incorporated herein. More specifically, a first method of forming such a sustained release device may be to dissolve a powder form of a biodegradable polymer into a solvent. The biological agent is added to the mixture and the mixture is dried to form a laminate material, and the laminate material is molded into the desired shape of the device. A second method is to melt the selected polymer into a molten state. The biological agent is then added to the molten polymer and the polymer is cooled under controlled conditions to create the desired shape of the device. In a third method, the biological agent may be incorporated within the depot of the present invention by simply soaking the selected depot polymer in solutions of the desired biological agents. Alternative methods for incorporating or impregnating the polymer with a biological agent have been described previously, for example, in U.S. Pat. Nos. 6,953,593, 6,946,146, 6,656,508, 6,541,033, 6,451,346, the contents of which are incorporated herein by reference. Many additional methods of preparation of a sustained-release formulation are known in the art and are disclosed in Remington's Pharmaceutical Sciences (18th ed.; Mack Publishing Company, Eaton, Pa., 1990), incorporated herein by reference. The present invention is not limited to any of the above methods of incorporating or impregnating a biological agent and may include any method understood in the art to incorporate or impregnate a polymer with a biological agent such that the polymer is in a sustained-release formulation.

The biological agent(s) for use in practicing the present invention include, but are not limited to, anti-inflammatory agents, antiviral agents, antibiotic agents and/or analgesic agents, or combinations thereof, each of which may be presented in a sustained-release formulation as a biological depot. As noted above, any such biological agent may be a molecule, cell or physical stimulus which promotes the intended biological response including, but not limited to, anti-inflammatory agents which are delivered by the methods of the present invention to promote a prolonged, sustained delivery of the agent(s) within the synovial joint. Such anti-inflammatory agents may be in any form such that administration of the entity promotes the desired anti-inflammatory response, including any molecule, cell or physical stimulus which positively effects the activity of an anti-inflammatory response. As discussed herein, a targeted inflammatory cytokine or protein related to the inflammatory response includes, but is not limited to, TNF-α, IL-1β, IL-6, IL-8, NF-κB, High Mobility Group Box 1 (HMG-B1), IL-2, IL-15, and MMPs while a specific anti-inflammatory cytokine or related protein which may promote an anti-inflammatory response includes, but is not limited to, IL-10, IL-4, IL-13 and TGF-β, as well as any other cytokine or pathway related protein which modulates the respective anti-inflammatory cytokine so as to impart an increase in the ability of reduce patient inflammation and pain within the synovial join.

In one embodiment of the present invention, the anti-inflammatory agent used in the methods of the present invention is an antagonist of TNF-α. TNF-α (also known as TNF or cachectin) is the prototype member of a large family of proteins with diverse functions. The TNF locus is located within the MHC gene cluster in humans (chromosome 6). More specifically, the gene encoding TNF-α is located within the MHC class IV cluster along with other family members, lymphotoxin-α (LT-α, formally TNF-β) and lymphotoxin-β (for a review, see Ruuls and Sedgwick, 1999, American J. Human Genetics 65:294-301). TNF-α is a soluble homotrimer of 17 kD protein subunits, with a 26 kD membrane bound precursor. TNF-α causes a pro-inflammatory cascade, resulting in tissue injury. For example, TNF-α induces an NF-κB-mediated survival and inflammatory pathway. Thus, it is now well known that TNF-α is an inflammatory cytokine; a cytokine secreted from macrophages and monocytes and which is involved in inflammatory diseases (causing a pro-inflammatory response leading to the breakdown of cartilage and bone), autoimmune diseases, bacterial infections, cancers and other degenerative diseases. This cytokine also functions as a signal transmitter in several pathological processes, such as necrosis and apoptosis, is involved in the process of promoting induction of an adhesion molecules, and an increase in the adherence of neutrophils and lymphocytes. To this end, TNF-α has been regarded as a useful target protein for a specific physiological treatment of rheumatoid arthritis, osteoarthritis, chondromalacia and any trauma or disorder wherein an inflammation response is elicited. Therefore, the present invention relates to, but it not limited to, use of an anti-cytokine agent which is an antagonist of TNF-α. Such a biological agent may be administered in devices known in the art, with a particular embodiment relating to the sustained-release of a TNF-α antagonist via depot implants within a synovial joint as described below. Suitable examples of such biological agents include but are not limited to adalimumab, infliximab, etanercept, pegsunercept (PEG sTNF-R1), sTNF-R1, CDP-870, CDP-571, CNI-1493, RDP58, ISIS 104838, 1?3-β-D-glucans, lenercept, PEG-sTNFRII Fc mutein, D2E7, afelimomab, and combinations thereof. A suitable TNF-a antagonist can also prevent or inhibit TNF-a synthesis and/or TNF-a release and includes compounds such as thalidomide, tenidap, and phosphodiesterase inhibitors, such as, but not limited to, pentoxifylline and rolipram. Additionally, the biological agent may also include any molecule, cell or physical stimulus which positively modulates the activity of the agent known to effect the inflammatory response. These agents can decrease pain through their actions as inhibitors or agonists of the release of pro-inflammatory molecules. For example, these substances can act by inhibiting or antagonizing expression or binding of cytokines or other molecules that act in the early inflammatory cascade, often resulting in the downstream release of prostaglandins and leukotrienes.

In another aspect of the present invention, the anti-cytokine agent is a TNF-a binding protein. One suitable such anti-cytokine agent is currently referred to as onercept. Any formulation comprising onercept, onercept-like agents, and derivatives are all considered acceptable to practice the methods of the present invention. Still other suitable biological agent includes dominant-negative TNF variants. A suitable dominant-negative TNF variant includes, but is not limited to, DN-TNF and including those described by Steed, et al. (2003, Science, 301:1895-1898). Still more embodiments include the use of a recombinant adeno-associated viral (rAAV) vector technology platform to deliver the oligonucleotides encoding inhibitors, enhancers, potentiators, neutralizers, or other modifiers. For example, in one embodiment a rAAV vector technology platform delivers the DNA sequence of a potent inhibitor of TNF-a. One suitable inhibitor is TNFR:Fc. Other anti-cytokine agents include antibodies, including but not limited to naturally occurring or synthetic, double chain, single chained, or fragments thereof, as discussed herein.

It is understood that TNF-a is both affected by upstream events which modulate its production and, in turn, affects downstream events. Alternative approaches to using such a compound is to exploit this fact, and antagonists are designed to specifically target TNF-a as well as molecules upstream, downstream and/or a combination thereof. Such approaches include, but are not limited, to modulating TNF-a directly, modulating kinases, inhibiting cell-signaling, manipulating second messenger systems, modulating kinase activation signals, modulating a cluster designator on an inflammatory cell, modulating other receptors on inflammatory cells, blocking transcription or translation of TNF-a or other targets in pathway, modulating TNF-a post-translational effects, employing gene silencing, or modulating interleukins. Anti-cytokine agents which inhibit TNF-a-post translational effects are useful in the invention. For example, the initiation of a TNF-a signaling cascade results in the enhanced production of numerous factors that subsequently act in a paracrine and autocrine fashion to elicit further production of TNF-a as well as other pro-inflammatory agents (IL-1β, IL-6, IL-8, HMG-B1). Extracellular TNF-a modifying anti-cytokine agents that act on the signals downstream of TNF-a are useful in treating systemic inflammatory diseases. Some of these anti-cytokine agents are designed to block other effector molecules while others block the cellular interaction needed to further induce their production, for example, integrins and cell adhesion molecules. Thus, the present invention also relates to a use of an anti-cytokine which antagonizes, for example, IL-1β, IL-6, IL-8 and HMG-B1.

In another embodiment of the present invention, the anti-inflammatory agent used in the methods of the present invention is an antagonist of interleukin 1-beta (IL-1β). Again, a particular embodiment of the methods of the present invention relates to the prolonged administration of an IL-1β antagonist via a sustained-release pharmaceutical depot implant within the knee joint. To this end, a portion of the present invention relates to an anti-cytokine agent which antagonizes the IL-1β-induced pro-inflammatory response. Interleukin 1-beta is a cytokine which shows a similar biological response compared to TNF-a. For example, certain inhibitors of this protein are similar to those developed to inhibit TNF-a. One such example is Kineret® (anakinra) which is a recombinant, non-glycosylated form of the human interleukin-1 receptor antagonist (IL-1Ra). Another way to incorporate use of IL-1Ra is to deliver an autologous blood serum, such as Orthokine®. Orthokine® serum is derived from the human patient's blood, which naturally contains IL-1Ra. The blood is removed, cultured in vitro to promote stimulation of monocytes and in turn increase production of IL-1Ra. The protein is extracted and then delivered back to the patient. In addition to use of IL-1Ra to antagonize IL-1, other suitable anti-cytokine agents may take the form as those described above regarding TNF-α. For example, a suitable anti-cytokine agent targeting IL-1 is an antibody against IL-1 which is effective in antagonizing the ability of IL-1 to interact with the type I or type II IL-1 receptor. Such an antibody includes, but is not limited to, AMG 108, a monoclonal antibody that blocks IL-1 activity. Any such antibody or antibody fragment, as discussed herein, will be useful to practice the methods of the present invention.

In another embodiment of the present invention, the anti-inflammatory agent used in the disclosed methods is an antagonist of interleukin-6 (IL-6). An antagonist of IL-6 for use in this portion of the invention may be any form of a biological agent as described herein. Interleukin-6 (IL-6) is a multifunctional cytokine that plays a central role in host defense due to its wide range of immune and hematopoietic activities and its potent ability to induce the acute phase response. Overexpression of IL-6 has been implicated in the pathology of a number of diseases including multiple myeloma, rheumatoid arthritis, Castleman's disease, psoriasis, and post-menopausal osteoporosis. To this end, selective antagonists of IL-6 activity will be useful as an anti-inflammatory in the methods of the present invention. A selective antagonist may be any such anti-cytokine agent as disclosed herein, including, but not limited to, an antibody against IL-6 such as a humanized anti-IL-6 mAb (MRA, tocilizumab, Chugai); or a human monoclonal antibody against IL-6, or an IL-6 ‘trap’, which would contain two soluble IL-6 receptors fused to an IgG molecule (i.e., Fc fragment) to produce an IL-6 dimer. Notwithstanding the foregoing, and as noted with other embodiments, the anti-cytokine agent which antagonizes IL-6 may come in any form disclosed herein, as well as any molecule, cell or physical stimulus which promotes the ability of such an agent to antagonize IL-6.

A further embodiment of the present invention contemplates administering an effective amount of an antagonist of interleukin-8 (IL-8) to the patient. Thus, the methods of promoting a sustained-release of a biological agent(s) within the knee joint will include administering to the subject an effective amount of an antagonist of a CXC chemokine involved in neutrophil infiltration. Interleukin-8, a 72 amino acid, tissue-derived peptide secreted by several types of cells in response to inflammatory stimuli, is a chemokine wherein 2 cysteines are separated by a single amino acid, thus referred to as a CXC chemokine. Chemokines of the CXC family show specificity for neutrophils, and to an extent, lymphocytes. Interleukin-8 is known to be directly involved in late cytokine activation of polymorphonuclear neutrophils (PMNs), leading to neutrophil activation and migration later in the inflammation cascade. To this end, an embodiment of the present invention relates to use of an anti-cytokine agent which acts as an antagonist of interleukin-8 (IL-8) to be administered to a patient to treat inflammation associated with a trauma or disorder of the knee. As noted with other embodiments, the anti-cytokine agent which antagonizes IL-8 may come in any form disclosed herein, as well as any molecule, cell or physical stimulus which promotes the ability of such an agent to antagonize IL-8.

Another embodiment of the methods of the present invention relate to utilizing a biological agent which is an antagonist of Nuclear Factor kappa B (NF-?B). Nuclear Factor kappa B is a transcription factor involved in cellular responses to stimuli such as stress, cytokines, free radicals, ultraviolet irradiation, and bacterial or viral antigens. NF-?B regulates inflammation, dendritic cell development and function, thymic selection and regulatory T cell production. There are five mammalian NF-?B family members which exist as homodimers or heterodimers: NF-?B1 (p50), NF-?B2 (p52), RelA (p65), RelB and c-Rel. All members share a Rel homology domain in their N-terminal region. RelA, RelB and c-Rel also have contain a trans-activation domain in their C-terminus. The NF-?B1 and NF-?B2 proteins are synthesized as larger precursors (p105 and p100, respectively) which undergo processing to generate the mature NF-?B subunits, p50 and p52. The processing of p105 and p100 is mediated by the ubiquitin/proteasome pathway and involves selective degradation of their C-terminal region containing ankyrin repeats. An active NF-?B transcription factor promotes expression of a number of genes, many of which participate through the canonical pathway to mediate the immune response, including cytokines such as TNF-α, IL-1β, IL-6 and GM-CSF and chemokines such as IL-8, RANTES, ICAN-1 and E-selectin. Again, the anti-cytokine agent which antagonizes NF-?B may come in any form disclosed herein, as well as any molecule, cell or physical stimulus which promotes the ability of such an agent to antagonize NF-?B. Additional anti-cytokine agents include, but are in no way limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, HMGB-1 mAb (Critical Therapeutics Inc.), anti-IL2R antibody (daclizumab, basilicimab), ABX (anti IL-8 antibody), HuMax IL-15 (anti-IL 15 antibody), NF-?B inhibitors such as for example glucocorticoids such as flucinolonone, antioxidants, such as dithiocarbamate, and other compounds such as sulfasalazine [2-hydroxy-5-[-4-[C2-pyridinylamino)sulfonyl]azo]benzoic acid], and clonidine.

A further embodiment of the present invention contemplates administering an effective amount of an inhibitor of a matrix metalloprotease (MMP) to the patient. Most MMP inhibitors are thiols or hydroxamates. Non-limiting examples of MMP inhibitors include TAPI-1 (TNF-a protease inhibitor) which blocks cleavage of cell surface TNF; TAPI-0, an analog of TAPI-1 that possesses similar efficacy in vitro; TAPI-2 which is inhibits both the activation-induced shedding of L-selectin from neutrophils, eosinophils, and lymphocytes and also inhibits phenylarsine oxide-induced L-selectin shedding; Ac-SIMP-1; Ac-SIMP-2; SIMP-1; SIMP-2; doxycycline; marimastat (British Biotech); cipemastat (Roche); and tissue inhibitor of metalloproteinases (TIMPs) which include TIMP-1, TIMP-2, TIMP-3 and TIMP-4. Synthetic inhibitors of MMPs generally contain a chelating group which binds the catalytic zinc atom at the MMP active site tightly. Common chelating groups include hydroxamates, carboxylates, thiols, and phosphinyls.

Another biological agent which may be considered when practicing the methods of the present invention relate to administering a pharmaceutically effective amount of interleukin-10 (IL-10). Interleukin-10 is an anti-inflammatory cytokine which will promote reduction of inflammation and pain. This non-limiting embodiment relates to administration of IL-10, or any biologically active fragment thereof as well as any molecule, cell or physical stimulus which promotes the ability IL-10 to impart the intended anti-inflammatory effect to the target patient by methods and drug delivery devices as described herein. Interleukin-10 is a homodimer with a molecular mass of 37 kDa. Each monomer consists of an identical 160 amino acid protein with a molecular mass of 18.5 kDa. Interleukin-10 is known as an important immunoregulatory cytokine which is expressed in numerous cell populations, with its main function seeming to be related to limitation and termination of inflammatory responses, as well as regulating the differentiation and proliferation of various immune cell types (for a review, see Asadullah, et al., 2003, Pharmacological Reviews 55(2):241-269). As noted by Asadullah, et al. (id.), it has been shown in several ex vivo studies that IL-10 can effectively block TNF-α, IL-1 and IL-8 by snivel macrophages and synoviocytes.

In an alternative embodiment, the biological agent of the present invention may be a growth factor. The growth factor may be an osteoinductive and/or cartilage forming protein or molecule that may be used alone or in combination with any of the above agents to stimulate or induce bone or cartilage growth within the joint. Platelet-derived growth factors (PDGFs), bone morphogenetic proteins (BMPs), insulin-like growth factors (IGFs), basic fibroblast growth factor (bFGF), cartilage derived morphogenetic protein (CDMP), and various other bone and cartilage regulatory proteins, such as CD-RAP or the like, are all growth factors that are successful in bone and cartilage regeneration. BMPs and CDMPs, in particular, induce new cartilage and bone formation though a signal cascade that, ultimately, leads to morphogenesis of precursor cells into bone or cartilage cells. CD-RAP is also known in the art to be a regulatory protein synthesized by chondrocytes involved in the formation of type II collagen and, ultimately, cartilage. To this end, BMPs, CDMPs, and CD-RAP may be contained within the depot of the present invention and released from the depot in accordance with the methods of the present invention such that the proteins or molecules induce the formation of bone and/or cartilage. Such formation of bone and/or cartilage leads to the treatment of the degeneration of cartilage and bone associated with osteoarthritis, chondromalacia, rheumatoid arthritis, or any other bone or cartilage degenerative condition.

Examples of such BMPs and CDMPs as discussed herein may include, but are not limited to, BMP-2, BMP-4, BMP-6, BMP-7, BMP-8, and CDMP-1. The BMPs or CDMPs may be available from Genetics Institute, Inc., Cambridge, Mass. and may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al. the contents of which are incorporated herein by reference. All osteoinductive factors are contemplated whether obtained as above or isolated from bone. Methods for isolating BMP from bone are described in U.S. Pat. No. 4,294,753 to Urist and Urist et al., 81 PNAS 371, 1984 the contents of which are incorporated herein by reference.

The present invention is not limited to the above embodiments of BMPs, CDMPs, and CD-RAP. Rather, any natural or synthetic BMP, CDMP or other osteoinductive or cartiliage producing protein or molecule is contemplated by the present invention such as, but not limited to, BMP-1, BMP-2, rhBMP-2, BMP-3, BMP-4, rhBMP-4, BMP-5, BMP-6, rhBMP-6, BMP-7 [OP-1], rhBMP-7, BMP-8, BMP-9, BMP-10, BMP-11, BMP-12, BMP-13, BMP-14, BMP-15, BMP-16, BMP-17, BMP-18, GDF-5 [CDMP-1], rhGDF-5. Additionally, the present invention may include, separately or in combination with any of the above embodiments, any other protein or molecule that induces bone or cartilage regeneration such as, but not limited to, platelet-derived growth factors (PDGFs), bone morphogenetic proteins (BMPs), insulin-like growth factors (IGFs), fibroblast growth factor (FGF), cartilage derived morphogenetic protein (CDMP), LIM mineralization proteins, transforming growth factors (TGF), fibroblast growth factor (FGF), and growth differentiation factors (GDF). A more detailed discussion as to how each of these growth factors and/or proteins induce bone and cartilage regeneration may be found in Rengachary, 2002, Neurosurg. Focus, 13:1-6; Reddi, 2001, Arthritis Res, 3:1-5; Varkey et al., 2004, Expert Opin. Drug Deliv., 1:19-36, the contents of which are incorporated herein by reference.

In additional embodiments of the invention, a biological agent may also include, but not be limited to, an antibiotic or an analgesic, or any combination thereof. Non limited examples of antibiotics include, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol and any combination thereof. Non-limiting suitable analgesics include morphine and naloxone, local anaesthetics (such as, for example, lidocaine, glutamate receptor antagonists, adrenoreceptor agonists, adenosine, canabinoids, cholinergic and GABA receptors agonists, and different neuropeptides. The above listed antibiotics and analgesics are not intended to be limiting. As such, an antibiotic or analgesic of the present invention may include any antibiotic or analgesic listed in any current or previous Physicians' Desk Reference. Moreover, a detailed discussion of different analgesics is provided in Sawynok et al., 2003, Pharmacological Reviews, 55:1-20, the content of which is incorporated herein by reference.

Additionally, suitable anti-inflammatory compounds for use in the present invention may include the compounds of both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, and triamcinolone. Mixtures of the above steroidal anti-inflammatory compounds can also be used.

Moreover, non-limiting examples of non-steroidal anti-inflammatory compounds that may be used in the present invention include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone. The various compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference. Finally, mixtures of these non-steroidal anti-inflammatory compounds may also be employed, as well as the pharmologically acceptable salts and esters of these compounds.

The biological agent(s) of the present invention may also be comprised of any molecule, cell, or physical stimulus which provides therapeutic or prophylactic relief for acute or chronic pain and/or inflammation associated with knee disorders including, but not limited to, osteoarthritis, rheumatoid arthritis, as well as known traumas associated with the tendons, ligaments and muscles in and around the knee joint. Such agents may include, but are not limited to, a small molecule, an oligonucleotide, an antibody or relevant antibody fragment as disclosed and further discussed herein, siRNA, as well as any factor in the form of a molecule, cell or physical stimulus which regulates expression of a gene of interest or effects stability or activity of the expressed transcript and/or translated protein, so as to modulate the target so as to provide a level of post-operative relief to the knee joint. To this end, these biological agent(s) may be any molecule, cell, or physical stimulus which provides therapeutic or prophylactic relief for acute or chronic pain and/or inflammation associated with any knee trauma or knee disorder, including traumas associated with the tendons, ligaments and muscles in and around the knee joint. In one embodiment, the injury or disorder is associated with an intraarticular ligament (e.g., the ACL and PCL). In another embodiment the trauma or disorder effects other ligaments of the knee joint (including but not limited to the lateral or medial collateral ligaments, as well as the patellar ligament). In other embodiments, the injuries or disorders relate to problems associated with the articular cartilage, such as osteoarthritis, chondromalacia, and rheumatoid arthritis. Another embodiment relates to treating meniscal injuries, such as meniscal tears. Additional embodiments include, but are not limited to, chondral fractures, traumas or injuries associated with the patella, just to name a few. Thus, the methods of the present invention may be utilized to deliver a biological agent to the knee joint area in a prolonged, sustained time frame to provide therapeutic or prophylactic treatment of any trauma or disorder of the knee while strategic placement of the depot implant(s) will provide for normal post-operative articulation of the knee joint.

Administration of an antibody as a biological agent is contemplated when practicing the methods of the present invention. An antibody may take one of numerous forms known in the art. Antibodies may take the form of any type of relevant antibody fragment, antibody binding portion, specific binding member, a non-protein synthetic mimic, or any other relevant terminology known in the art which refers to an entity which at least substantially retains the binding specificity/neutralization activity. Thus, the term “antibody” as used in any context within this specification is meant to include, but not be limited to, any specific binding member, immunoglobulin class and/or isotype (e.g., IgG1, IgG2, IgG3, IgG4, IgM, IgA, IgD, IgE and IgM); and biologically relevant fragment or specific binding member thereof including, but not limited to, Fab, F(ab′)2, Fv, and scFv (single chain or related entity), which are capable of binding to the respective targeted cytokine. Therefore, it is well known in the art, and is included as review only, that an “antibody” refers to a glycoprotein comprising at least two heavy (H) chains and two light (L) chains inter-connected by disulfide bonds, or an antigen binding portion thereof. A heavy chain is comprised of a heavy chain variable region (VH) and a heavy chain constant region (CH1, CH2 and CH3). A light chain is comprised of a light chain variable region (VL) and a light chain constant region (CL). The variable regions of both the heavy and light chains comprise a framework (FW) and complementarily determining regions (CDR). The four FW regions are relatively conversed while CDR regions (CDR1, CDR2 and CDR3) represent hypervariable regions and are arranged from NH₂ terminus to the COOH terminus as follows: FW1, CDR1, FW2, CDR2, FW3, CDR3, FW4. The variable regions of the heavy and light chains contain a binding domain that interacts with an antigen while, depending of the isotype, the constant region(s) may mediate the binding of the immunoglobulin to host tissues or factors. That said, also included in the working definition of “antibody” are chimeric antibodies, humanized antibodies, a recombinant antibody, as human antibodies generated from a transgenic non-human animal, as well as antibodies selected from libraries using enrichment technologies available to the artisan. Antibody fragments are obtained using techniques readily known and available to those of ordinary skill in the art, as reviewed below. Therefore, an “antibody” is any such entity or specific binding member, which specifically binds to the respective target cytokine so as to inhibit the ability of the cytokine to impart a normal inflammatory response. Any such entity is a candidate for therapeutic applications disclosed herein. Therefore, the term “antibody” describes an immunoglobulin, whether natural or partly or wholly synthetically produced; any polypeptide or protein having a binding domain which is, or is substantially homologous to, an antibody binding domain. These can be derived from natural sources, or they may be partly or wholly synthetically produced. Examples of antibodies are the immunoglobulin isotypes and their isotypic subclasses; fragments which comprise an antigen binding domain such as Fab, scFv, Fv, dAb, Fd and diabodies, as discussed without limitation, infra. It is known in the art that it is possible to manipulate monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarily determining regions (CDRs), of an antibody to the constant regions, or constant regions plus framework regions, of a different immunoglobulin. A hybridoma or other cell producing an antibody may be subject to genetic mutation or other changes, which may or may not alter the binding specificity of antibodies produced. Antibodies can be modified in a number of ways, and the term “antibody” should be construed as covering any specific binding member or substance having a binding domain with the required specificity. Thus, this term covers antibody fragments, derivatives, functional equivalents and homologues of “antibody” including any polypeptide comprising an immunoglobulin binding domain, whether natural or wholly or partially synthetic. Such an entity may be a binding fragment encompassed within the term “antigen-binding portion” or “specific binding member” of an antibody including, but not limited to, (i) a Fab fragment, a monovalent fragment consisting of the VL, VH, CL and CH domains; (ii) a F(ab′)2 fragment, a bivalent fragment comprising two Fab fragments linked by a disulfide bridge at the hinge region; (iii) a Fd fragment consisting of the VH and CH domains; (iv) a Fv fragment consisting of the VL and VH domains of a single arm of an antibody (v) a dAb fragment, which comprises a VH domain; (vi) an isolated complementarily determining region (CDR); (vii) a ‘scAb’, an antibody fragment containing VH and VL as well as either CL or CH; and (viii) artificial antibodies based upon protein scaffolds, including but not limited to fibronectin type III polypeptide antibodies (e.g., see U.S. Pat. No. 6,703,199, issued to Koide on Mar. 9, 2004 and PCT International Application Publication No. WO 02/32925). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)).

Polyclonal or monoclonal antibodies for use in the disclosed treatment methods may be raised by known techniques. Monospecific murine (mouse) antibodies showing specificity to a conformational epitope of a target of choice may be purified from mammalian antisera containing antibodies reactive against this region, or may be prepared as monoclonal antibodies using the technique of Kohler and Milstein (1975, Nature 256: 495-497). Monospecific antibody as used herein is defined as a single antibody species or multiple antibody species with homogenous binding characteristics. Hybridoma cells are produced by mixing the splenic lymphocytes with an appropriate fusion partner, preferably myeloma cells, under conditions which will allow the formation of stable hybridomas. The splenic antibody producing cells and myeloma cells are fused, selected, and screened for antibody production. Hybridoma cells from antibody positive wells are cloned by a technique such as the soft agar technique of MacPherson (1973, Soft Agar Techniques, in Tissue Culture Methods and Applications, Kruse and Paterson, Eds, Academic Press). Monoclonal antibodies are produced in vivo by injecting respective hydridoma cells into pristine primed mice, collecting ascite fluid after an interval of time, and prepared by techniques well known in the art.

Beyond species specific monoclonal antibodies described above, the antibodies of the present invention may also be in the form of a “chimeric antibody”, a monoclonal antibody constructed from the variable regions derived from say, the murine source, and constant regions derived from the intended host source (e.g., human; for a review, see Morrison and Oi, 1989, Advances in Immunology, 44: 65-92). The variable light and heavy genes from the rodent (e.g., mouse) antibody are cloned into a mammalian expression vector which contains an appropriate human light chain and heavy chain coding region, respectively. These heavy and light “chimeric” expression vectors are cotransfected into a recipient cell line and selected and expanded by known techniques. This cell line may then be subjected to known cell culture techniques, resulting in production of both the light and heavy chains of a chimeric antibody Such chimeric antibodies have historically been shown to have the antigen-binding capacity of the original rodent monoclonal while significantly reducing immunogenicity problems upon host administration.

A logical improvement to the chimeric antibody is the “humanized antibody,” which arguably reduces the chance of the patient mounting an immune response against a therapeutic antibody when compared to use of a chimeric or full murine monoclonal antibody. The strategy of “humanizing” a murine Mab is based on replacing amino acid residues which differ from those in the human sequences by site directed mutagenesis of individual residues or by grafting of entire complementarily determining regions (Jones et al., 1986, Nature 321: 522-526). This technology is again now well known in the art and is represented by numerous strategies to improve on this technology; namely by implementing strategies including, but not limited to, “reshaping” (see Verhoeyen, et al., 1988, Science 239: 1534-1536), “hyperchimerization” (see Queen, et al., 1991, Proc. Natl. Acad. Sci. 88:2869-2873) or “veneering” (Mark, et al., 1994, Derivation of Therapeutically Active Humanized and Veneered anti-CD18 Antibodies Metcalf end Dalton, eds. Cellular Adhesion: Molecular Definition to Therapeutic Potential. New York: Plenum Press, 291-312). These strategies all involve, to some degree, sequence comparison between rodent and human sequences to determine whether specific amino acid substitutions from a rodent to human consensus is appropriate. Whatever the variations, the central theme involved in generating a humanized antibody relies on CDR grafting, where these three antigen binding sites from both the light and heavy chain are effectively removed from the rodent expressing antibody clone and subcloned (or “grafted”) into an expression vector coding for the framework region of the human antibody. Therefore, a “humanized antibody” is effectively an antibody constructed with only murine CDRs (minus any additional improvements generated by incorporating one or more of the above mentioned strategies), with the remainder of the variable region and all of the constant region being derived from a human source.

Yet another improvement over re-engineered antibodies as reviewed above is the generation of fully human monoclonal antibodies. The first involves the use of genetically engineered mouse strains which possess an immune system whereby the mouse antibody genes have been inactivated and in turn replaced with a repertoire of functional human antibody genes, while leaving other components of the mouse immune system unchanged. Such genetically engineered mice allow for the natural in vivo immune response and affinity maturation process which results in high affinity, fully human monoclonal antibodies This technology is again now well known in the art and is fully detailed in various publications including, but not limited to, U.S. Pat. Nos. 5,939, 598; 6,075,181; 6,114,598; 6,150,584 and related family members (assigned to Abgenix, disclosing their XenoMouse technology); as well as U.S. Pat. Nos. 5,545,806; 5,569,825; 5,625,126; 5,633,425; 5,789,650; 5,877, 397; 5,661,016; 5,814,318; 5,874,299; and 5,770,429 (assigned to GenPharm International and available through Medarex, under the umbrella of the “UltraMab Human Antibody Development System”). See also a review from Kellerman and Green (2002, Curr. Opinion in Biotechnology 13: 593-597).

Finally, techniques are available to the artisan for the selection of antibody fragments from libraries using enrichment technologies including, but not limited to, phage display, ribosome display (Hanes and Pluckthun, 1997, Proc. Nat. Acad. Sci. 94: 4937-4942), bacterial display (Georgiou, et al., 1997, Nature Biotechnology 15: 29-34) and/or yeast display (Kieke, et al., 1997, Protein Engineering 10: 1303-1310) may be utilized as alternatives to previously discussed technologies to select single chain antibodies which specifically bind to target cytokine. Single-chain antibodies are selected from a library of single chain antibodies produced directly utilizing filamentous phage technology. Phage display technology is known in the art (e.g., see technology from Cambridge Antibody Technology (CAT)) as disclosed in U.S. Pat. Nos. 5,565,332; 5,733,743; 5,871,907; 5,872,215; 5,885,793; 5,962,255; 6,140,471; 6,225,447; 6,291650; 6,492,160; 6,521,404; 6,544,731; 6,555,313; 6,582,915; 6,593,081, as well as other U.S. family members, or applications which rely on priority filing GB 9206318, filed 24 May 1992; see also Vaughn, et al. 1996, Nature Biotechnology 14: 309-314). Single chain antibodies may also be designed and constructed using available recombinant DNA technology, such as a DNA amplification method (e.g., PCR), or possibly by using a respective hybridoma cDNA as a template. Single-chain antibodies can be mono-or bispecific; bivalent or tetravalent. A nucleotide sequence encoding a single-chain antibody can be constructed using manual or automated nucleotide synthesis, cloned into an expression construct using standard recombinant DNA methods, and introduced into a cell to express the coding sequence, as described below.

The term “recombinant human antibody” represents a viable subset of “antibodies” generated by various means of recombinant DNA technology and non-human transgenics that are well known in the art. Such methodology is utilized to generate an antibody from one or the following origins: (i) a scFv or alternative antibody isolated from a combinatorial human antibody library; (ii) a partial or complete antibody generated from a respective expression vector stably or transiently transfected into a host cell, preferably a mammalian host cell; and/or (iii) an antibody isolated from a non-human transgenic animal which contains human immunoglobulin genes, or by any other known methodology which relies of the recombinant ‘mixing and matching’ of human immunoglobulin gene sequences to other DNA sequences in order to generate the human recombinant antibody of interest.

In a similar manner, a gene encoding a target protein disclosed herein, either in a normal or in a mutant form, can be down regulated through the use of antisense oligonucleotides directed against the gene or its transcripts. A similar strategy can be utilized as discussed herein in connection with an antibody raised against such a target protein. For a particularly valuable review of the design considerations and use of antisense oligonucleotides see Uhlmann et al., Chemical Reviews, 1990, 90(4):543-584, the disclosure of which is hereby incorporated by reference. The antisense oligonucleotides of the present invention may be synthesized by any of the known chemical oligonucleotide synthesis methods. Such methods are generally described, for example, in Winnacker, Chirurg 1992, 63(8), Supp.145-149 (German).

Since the complete nucleotide synthesis of DNA complementary to any of the target genes contemplated herein is known, the mRNA transcript of the cDNA sequence is also known. As such, antisense oligonucleotides hybridizable with any portion of such transcripts may be prepared by oligonucleotide synthesis methods known to those skilled in the art. While any length oligonucleotide may be utilized in the practice of the invention, sequences shorter than 12 bases may be less specific in hybridizing to the target mRNA, may be more easily destroyed by enzymatic digestion, and may be destabilized by enzymatic digestion. Hence, oligonucleotides having 12 or more nucleotides are preferred. Long sequences, particularly sequences longer than about 40 nucleotides, may be somewhat less effective in inhibiting translation because of decreased uptake by the target cell. Thus, oligomers of 12-40 nucleotides are preferred, more preferably 15-30 nucleotides, most preferably 18-26 nucleotides. Sequences of 18-24 nucleotides are most particularly preferred.

In one embodiment, the antisense therapy may be accomplished by siRNA or shRNA treatment. SiRNAs are typically short (19-29 nucleotides), double-stranded RNA molecules that cause sequence-specific degradation of complementary target mRNA known as RNA interference (RNAi) (Bass, Nature, 2001, 411:428-429). Accordingly, in some embodiments, the siRNA molecules comprise a double-stranded structure comprising a sense strand and an antisense strand, wherein the antisense strand comprises a nucleotide sequence that is complementary to at least a portion of a desired nucleic acid sequence and the sense strand comprises a nucleotide sequence that is complementary to at least a portion of the nucleotide sequence of said antisense region, and wherein the sense strand and the antisense strand each comprise about 19-29 nucleotides.

The siRNA molecules targeted to desired sequence can be designed based on criteria well known in the art (e.g., Elbashir et al., EMBO J., 2001, 20(23):6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; the siRNA molecule preferably should comprise two nucleotide overhangs (preferably TT) at each 3′ terminus; the target segment preferably should be in the ORF region of the target mRNA and preferably should be at least 75 bp after the initiation ATG and at least 75 bp before the stop codon; and the target segment preferably should not contain more than 16-17 contiguous base pairs of homology to other coding sequences.

Based on some or all of these criteria, siRNA molecules targeted to desired sequences can be designed by one of skill in the art using the aforementioned criteria or other known criteria (e.g., Gilmore et al., 2004, J. Drug Targeting 12(6):315-40; Reynolds et al., 2004, Nature Biotechnol. 22(3):326-30; Ui-Tei et al., 2004, Nucleic Acids Res. 32(3):936-948). Such criteria are available in various web-based program formats useful for designing and optimizing siRNA molecules (e.g., Sidesign Center at Dharmacon; BLOCK-IT RNAi Designer at Invitrogen; siRNA Selector at WISTAR Insitute; siRNA selection program at Whitehead Institute; siRNA Design at Integrated DNA Technologies; siRNA Target Finder at Ambion; AND siRNA Target Finder at Genscript). Accordingly, a person of skill in the art may just find suitable siRNA sequences by entering the desired template sequence into one or more of the software programs listed above.

In one embodiment, the siRNA molecules targeted may be to desired sequences can be produced in vitro by annealing two complementary single-stranded RNA molecules together (one of which matches at least a portion of a desired nucleic acid sequence) (e.g., U.S. Pat. No. 6,506,559) or through the use of a short hairpin RNA (shRNA) molecule which folds back on itself to produce the requisite double-stranded portion (Yu et al., Proc. Natl. Acad. Sci. USA, 2002, 99:6047-6052). Such single-stranded RNA molecules can be chemically synthesized (e.g., Elbashir et al., 2001, Nature 411(6836):494-8) or produced by in vitro transcription using DNA templates (e.g., Yu et al., 2002, Proc. Natl. Acad. Sci. USA 99:6047-6052). When chemically synthesized, chemical modifications can be introduced into the siRNA molecules to improve biological stability. Such modifications include phosphorothioate linkages, fluorine-derivatized nucleotides, deoxynucleotide overhangs, 2′-O-methylation, 2′-O-allylation, and locked nucleic acid (LNA) substitutions (Dorset and Tuschl, 2004, Nat. Rev. Drug Discov. 3:318); Gilmore et al., 2004, J. Drug Targeting 12(6):315-40).

Administration of a siRNA as a biological agent is contemplated when practicing the methods of the present invention. More specifically, siRNA molecules targeted to desired target sequences can be released from the implanted pharmaceutical depot and taken up into lymphocytes within the knee joint in order to inhibit expression of a target gene encoding an inflammatory-related cytokine, chemokine, etc. As discussed herein, a targeted inflammatory cytokine or protein related to the inflammatory response includes, but is not limited to, TNF-α, IL-1β, IL-6, IL-8, NF-κB, High Mobility Group Box 1 (HMG-B1), IL-2, and IL-15, while a specific anti-inflammatory cytokine or related protein which may promote an anti-inflammatory response includes but is not limited to IL-10, IL-4, IL-13 and TGF-β, as well as any other cytokine or pathway related protein which modulates the respective anti-inflammatory cytokine so as to impart an increase in the ability to reduce inflammation and pain within a joint such as the knee joint.

As noted above, the composition of the depot 55, the tether 60, and the cap 70 of the present invention may include any of the above natural or synthetic, biodegradable, biostable polymers, or combinations thereof. Additionally, the depot 55 may be impregnated or incorporated, by methods discussed above, with any one or combination of biological agents, as discussed above, to form a sustained-release device. Once implanted within the subject, in one embodiment the depot 55 polymer begins to degrade within the body. Such degradation causes the gradual release of the biological agent within the depot 55 in accordance with the half life of the polymer composition of the depot 55. Accordingly, the selection of the polymers for the composition of the depot 55 are based, inter alia, upon an evaluation of the desired doses of the biological agent to be released, the time line of release, and half life of single polymer or half lives of the combinations of polymers selected for the depot 55. Additionally, because the tether 60 and cap 70 function to couple the depot 55 to the synovial membrane 45 on the interior side 85 of the synovial joint capsule 1, the half lives of the polymers are also pertinent to determining which polymer should be used in creating the tether 60 and the cap 70. In one embodiment, the polymer comprising the depot 55 is of such a material as to avoid unintended reactions with the biological agent, and is preferably biocompatible with the synovial joint (e.g., where the dosage form is implanted, it is substantially non-reactive with respect to a subject's body or body fluids). Generally, the biological agent(s) of the present invention are designed to be delivered to the synovial joint for at least 10, 20, 30, 100 days or at least 4 months, or at least 12 months or more, as required. Specific ranges of amount of biological agent delivered will vary depending upon, for example, the potency and other properties of the agent used and the therapeutic requirements of the subject. Accordingly, the invention is not limited to the above time frames. Finally, the depot is not limited to the above embodiment but may be any sustained release formulation that the depot 55 is designed to gradually release a biological agent to a targeted region.

In operation, as illustrated in FIGS. 2C, 7B and 8B the depot 55 is surgically inserted by a minimally invasive means through an incision 95 created in a synovial membrane wall 45, such as, but not limited to, a knee joint, such that the depot 55 is on an interior side 85 of the joint capsule membrane 45. The tether 60 extends back through the incision 95 in the membrane 45 such that the distal end 65 of the tether 60 is exposed to the exterior side 75 of the membrane 45. The cap 70 is then secured, in any of the above embodiments, to the distal end 65 of the tether 60 such that the cap 70 is retained on the tether 60 and on the exterior side 75 of the membrane 45. Additionally, in one embodiment, because the length of the tether 60 is approximately the same length as the membrane 45, the action of snap fitting the cap 70 to the tether 60 couples the depot 55 to the membrane 45 on the interior side of the joint capsule 85 such that the depot is secured to the synovial joint 1 without interfering with normal joint articulation. In another embodiment, the composition of the cap 70 and the tension created between the cap and the depot by the tether functions to seal the incision 95 created in the membrane 45 such that neither synovial fluid nor the biological agent is able to leak outside of the synovial joint 1. As illustrated in FIG. 6, the depot may be implanted on the synovial membrane 45 of the joint such that the device 51 does not interfere with normal articulation of the joint. Surgical insertion of the depot can also include making a hole in the synovial membrane by spreading apart the synovial membrane using a blunt instrument and inserting the depot through that hole. In any case, once implanted, the depot begins to degrade releasing the impregnated biological agents in accordance with the above.

The phrase “minimally invasive” as used herein, refers to non-operative means of incorporating a pharmaceutical depot into a joint. For example, the depot may be implanted non-operatively in that the patient is under anesthesia local to the joint and the depot is implanted through a cannula, as discussed below. The definition of minimally invasive is not limited to this embodiment and may include any non-operative or outpatient procedure understood in the art.

One advantage to the present invention is that the device is capable of being implanted into the joint while eliminating the risk of infection associated with prior methods of drilling. A second advantage to the present invention is the device is capable of being implanted into the joint while eliminating the risk of compromising the structural integrity of bone structure and ligament structure within the synovial joint. A third advantage is that use of a sustained-release drug delivery device obviates the need for regular dosing by the patient, thus increasing patient compliance with a prescribed therapeutic regimen, and in particular compliance with a prophylactic regimen prescribed prior to the onset of symptoms with minimal invasiveness into the synovial capsule. Long-term delivery from an implanted dosage form provides an effective and less expensive method of providing care to subjects suffering from trauma and/or acute or chronic disorders of the synovial joint. A fourth advantage of the invention is that the biological agent can be delivered continuously with accuracy and precision and at low quantities as to permit long-term use, especially as an anti-inflammatory, antibiotic and/or analgesic without creating unnecessary damage to the synovial joint during implantation.

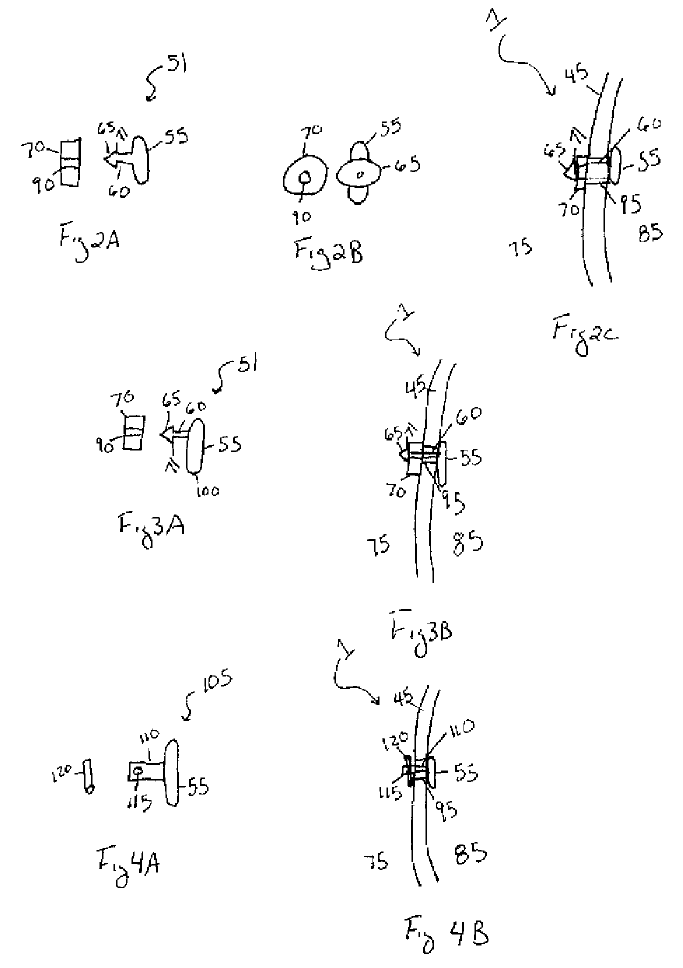

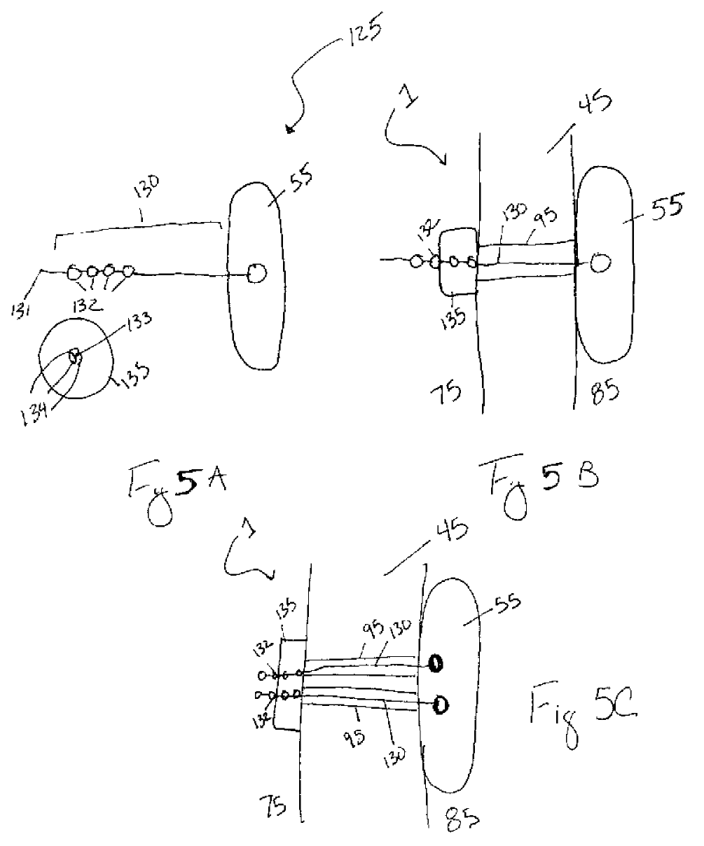

Referring to FIGS. 3A and 3B, a second embodiment of the present invention is illustrated. More specifically, the depot 55 may be a rod, as illustrated in FIG. 3A, a disc, a cylinder, or any other shape understood in the art to act as a sustained-release drug device or depot. The depot 55 may take the form of any solid, biodegradable, natural or synthetic polymer or combinations thereof, as discussed above, and may contain at least one biological agent, as discussed above. In this second embodiment the tether 60 may be, but is not limited to, a rod-like structure extending from the depot 55 at approximately a perpendicular angle relative to the depot 55. However, the tether 60 is not centered on the depot 55 as in the first embodiment. Rather the tether 60 extends from the depot 55 at an end of the depot 55. The length of the tether 60 may be of any length necessary for implantation into the synovial joint, however, as in the first embodiment, the tether is preferably the approximate width of the synovial membrane 45, as illustrated in FIG. 3B, such that the end distal of the tether 65 is exposed on the exterior side 75 of the synovial membrane 45 and the depot 55 is coupled to the synovial membrane 45 on the interior side of the synovial joint 1. In this second embodiment, the tether 60 is adapted at its distal end to be secured to a disc-shaped cap 70 through a snap fit or press fit mechanism, as illustrated in the embodiments of FIGS. 3A, 7A and 8A. Alternatively, the cap and tether may be coupled through a threadingly engagable methods such as that disclosed above. Finally, both the tether 60 and the cap 70 may be comprised of a solid, biodegradable or non-biodegradable, natural or synthetic polymer or any combination thereof, as discussed above, and may be comprised of the same material as the depot 55 or material distinguishable from the depot 55.

The second embodiment is advantageous in that the distance between the tether 60 and the opposing end 100 of the depot 55 is greater. Accordingly, the depot 55 may be implanted into the synovial joint 1 with greater ease. Moreover, a small incision 95 in the membrane 45 is all that is required to implant the depot 55 within the synovial joint 1. This, in turn, reduces the risk of any adverse consequences stemming from implantation of the device such as, but not limited to, leakage of the synovial fluid, infection, or any further complications.

Referring to FIG. 4A and FIG. 4B a third embodiment of the present invention is illustrated. In the third embodiment, the depot 55 may be a rod, as illustrated in FIG. 4A, a disc, a cylinder, or any other shape understood in the art to act as a sustained-release drug device or depot. The depot 55 may take the form of any solid, biodegradable, natural or synthetic polymer or combinations thereof, as discussed above, and may contain at least one biological agent, as discussed above. The tether 110 of the third embodiment may be, but is not limited to, a rod-like or flat structure extending from the depot 55 at approximately a perpendicular angle relative to the depot 55. The length of the tether 110 may be of any length necessary for implantation into the synovial joint, however, as in the first embodiment, the tether is preferably the approximate width of the synovial membrane 45, as illustrated in FIG. 4B, such that the end distal of the tether 65 is exposed on the exterior side 75 of the synovial membrane 45 and the depot 55 is coupled to the synovial membrane 45 on the interior side 85 of the synovial joint 1. In the third embodiment, the tether 110 is adapted at its distal end to receive a rod-shaped cap 120. More specifically, the distal end of the tether 110 has a hole or slot 115, the axis of which is perpendicular to the longitudinal axis of the tether 110. The diameter of the hole 115 is approximately equivalent to the diameter of the cap 120. The cap may contain a plurality of ridges (not illustrated) coaxially surrounding the diameter of the cap 120 and approximately centered thereon. The circumference of the ridges may be slightly larger than the diameter of the hole 115 of the tether 110 such that the cap may be snap fit or press fit into the hole 115. Alternatively, the ridges of the cap 120 and the hole 115 of the tether may be adapted such that the cap is threaded into the hole 115 of the tether 110. Finally, both the tether 110 and the cap 120 may take the form of a solid, biodegradable or non-biodegradable, natural or synthetic polymer or any combination thereof, as discussed above, and may be comprised of the same material as the depot 55 or material distinguishable from the depot 55.

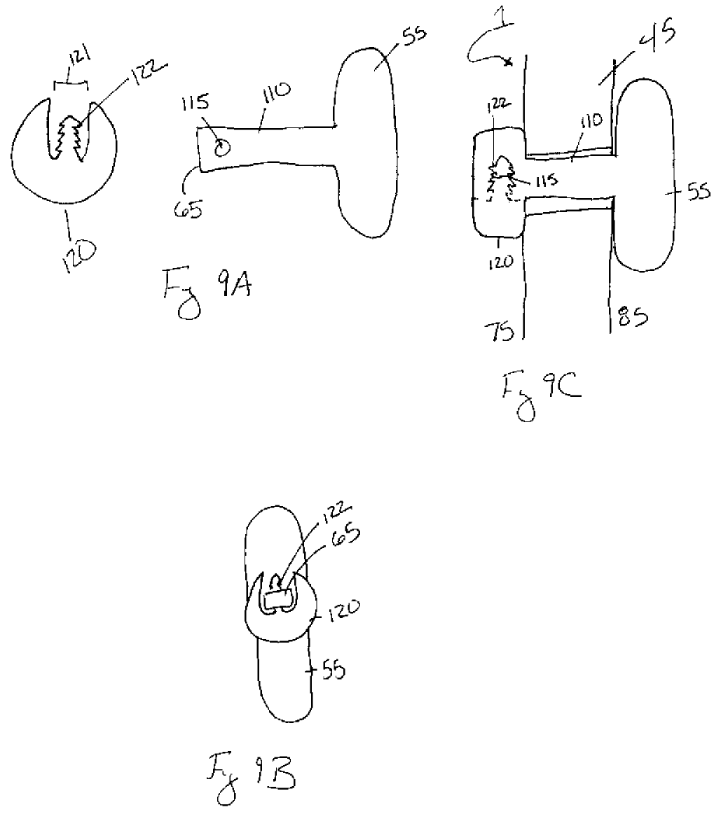

The shape of the cap, as illustrated in FIGS. 4A and 4B is not limited to a rod-like structure. Referring to FIGS. 9A, 9B, and 9C, the cap 120 may be in a disk-like shape or hemispherical shape with a recess 121. The recess 121 may contain at least one ridge 122 extending from the recess wherein the ridge(s) are slightly larger than the diameter of the hole 115 of the tether. To this end, as illustrated in FIGS. 9B and 9C, the hole 115 of the tether 110 is adapted to receive the ridge(s) 122 of the cap 120 such that the ridge(s) 122 of the cap 120 are press fit or snapped into the hole 115 of the tether 110. Because the circumference of the ridges 122 is slightly larger than the circumference of the hole 115, the ridge(s) 122 frictionally secure the cap 120 to the tether 110. As illustrated in FIG. 9C, length of the tether 110 is the approximate width of the synovial membrane 45 such that the tether 110 may be exposed on the exterior side 75 of the synovial membrane 45 and the depot 55 is coupled to the synovial membrane 45 on the interior side 85 of the synovial joint 1.

In operation, the cap 120 may be inserted into the hole 115 of the tether 110 such that, when the device is implanted into a synovial joint, the cap 120 is secured to the tether 115 and coupled to the membrane 45 on the exterior side of the synovial joint capsule 1. Additionally, because the length of the tether 110 is approximately the same length as the membrane 45, the insertion of the cap 120 into the hole 120 of the tether 110 couples the depot 55 to the membrane 45 on the interior side of the synovial joint 1 and secures the depot 55 to the synovial joint 1 without interfering with normal joint articulation.

An advantage of this third embodiment is that the depot may be implanted within the synovial joint with greater ease. Accordingly, the structural integrity of the membrane 45 is maintained and there is less risk of infection and/or synovial fluid or biological agent leaking from the synovial joint.

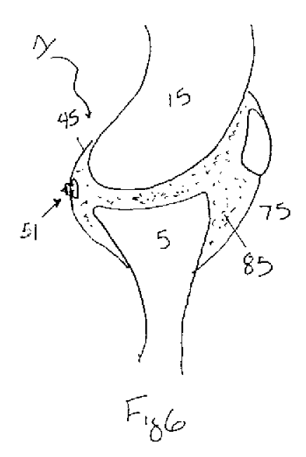

Referring to FIGS. 5A, 5B, and 5C a fourth embodiment of the present invention is illustrated. In the fourth embodiment, the depot 55 may be a rod, as illustrated in FIG. 5A, a disc, a cylinder, or any other shape understood in the art to act as a sustained-release drug device or depot. The depot 55 may be composed of any solid, biodegradable, natural or synthetic polymer or combinations thereof, as discussed above, and may contain at least one biological agent, as discussed above. The tether 130 of the fourth embodiment may be, but is not limited to, at least one suture extending from the depot 55. The length of the tether 110 may be of any length necessary for implantation into the synovial joint, however, as in the first embodiment, the tether is at least the approximate width of the synovial membrane 45, as illustrated in FIG. 5C, such that the end distal of the tether 130 is exposed on the exterior side 75 of the synovial membrane 45 and the depot 55 is coupled to the synovial membrane 45 on the interior side 85 of the synovial joint 1. At a distal end 131 of the tether 130 is a plurality of beads or knots 132. The beads or knots 132 are adapted to secure the tether, and ultimately the depot, to a cap 135. The cap 135 may be a disc, as illustrated in FIG. 5A, a rod, a cylinder, or any other similar shape. A hole 133 extends through the center of the cap 135 wherein the circumference of the hole approximates the circumference of the beads or knots 132. The hole 133 may further be comprised of a plurality of flaps 134 wherein the flaps function as a one-way valve. To this end, the flaps 134 facilitate securing the tether 130 and depot 55 to the synovial membrane by allowing the beads or knots 132 to pass through the hole 133 in the cap 135 in one direction, such as during implantation, but preventing the beads or knots 132 from passing back through the hole 133 in the opposite direction. As noted above, both the tether 110 and the cap 135 may take the form of a solid, biodegradable or non-biodegradable, natural or synthetic polymer or any combination thereof and may be comprised of the same material as the depot 55 or material distinguishable from the depot 55.

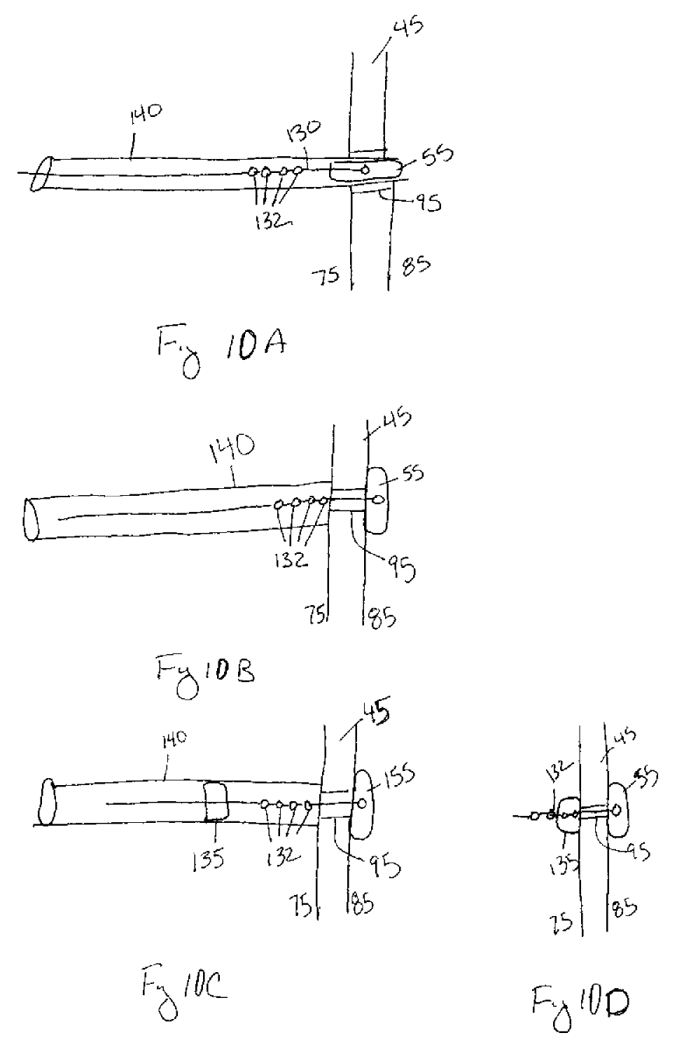

Referring to FIGS. 10A-10D, in operation, the depot 55 is surgically inserted through minimally invasive means. More specifically, referring to FIG. 10A, at least one incision 95 may be created in a synovial membrane wall 45, such as a knee joint, and a hollow cannula 140 is inserted therein. The cannula 140 is inserted such that the depot 55 may be threaded through the cannula 140 to an interior side 85 of the synovial membrane 45. The tether 130 may extend from the depot in accordance with the above such that the tether is coaxial with the cannula 140 and an operator may manipulate the passage of the depot 55 through the cannula 140 by the tether 130. Once the depot is within the synovial capsule 1, as illustrated in FIG. 10B, the cannula 140 may be retracted back through the incision 95. The operator may apply slight counter pressure to the tether such that the depot 55 is secure against the interior side 85 of the synovial membrane 45 and substantially seals the incision 95. Referring to FIG. 10C, the cap 135 may be threaded through the cannula 140 such that the tether 130 passes through the hole 133 in the cap 135. Ultimately, the beads or knots 132 are also threaded through the hole 133 and flaps 135 such that the action of the flaps 135 secures the cap to the exterior side 75 of the synovial membrane 45 and secures the depot to the interior side 85 of the synovial membrane 45. The operator may then cut the tether such that at least one bead or knot 132 extends through the hole 133 in the cap 135 and secures the cap to the depot. In a further embodiment, the biocompatibility of the cap 135 and the tension created by the tether 130 holding both the cap 135 and depot 55 against the membrane wall 45 functions to seal the incision 95 created in the membrane 45 such that neither synovial fluid nor the biological agent is able to leak outside of the synovial joint 1. As illustrated in FIG. 6, the device may be implanted such that it does not interfere with normal joint articulation. The invention, however, is not limited to this embodiment and may be comprised of any material understood in the art to seal an incision in soft tissue.

The device 125 of the fourth embodiment is not limited to only one tether and may be comprised of more than one tether 130. As illustrated in FIG. 5C, the tether may be comprised of, but not limited to, two or more tethers 130 wherein the tethers 130 may be threaded through one or a plurality of incisions 95. To this end, one or more incisions 95 would be made into the membrane 45 and each tether 130 may be threaded through its respective incision 95 and secured to the cap 135 in accordance with the above minimally invasive method.

As noted above, the minimally invasive method is applicable to any and all embodiments discussed above. To this end, additional instruments understood in the art to facilitate implantation through a cannula may be used in conjunction with the cannula and may vary in accordance with the particular embodiment of the device implanted. Furthermore, one can make a hole in the synovial membrane using a blunt instrument to pry or spread apart the membrane.

An advantage of this fourth embodiment is that the depot may be implanted within the synovial joint with only a small incision or hole in the synovial membrane 45. Accordingly, the structural integrity of the membrane 45 is maintained and there is less risk of infection and/or synovial fluid or biological agent leaking from the synovial joint.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

We claim:

1. An apparatus for providing prolonged treatment to a joint comprising:
a depot wherein the depot contains a biological agent;
a tether wherein the tether extends from the depot; and
a cap wherein the tether can be secured to the cap such that the depot is secured to a membrane of a joint capsule of the joint.

2. The apparatus of claim 1, wherein the cap is secured to the tether through at least one ridge extending from the cap wherein the ridge snap fits the cap to at least one groove within a slot of the tether.

3. The apparatus of claim 1, wherein the tether is secured to the cap through at least one ridge extending from the tether wherein the ridge snaps fits the tether to at least one groove within a slot of the cap.

4. The apparatus of claim 1, wherein the cap is secured to the tether through at least one ridge extending from the cap wherein the ridge threadingly engages a groove within a slot of the tether.

5. The apparatus of claim 1, wherein the tether is secured to the cap through at least one ridge extending from the tether wherein the ridge threadingly engages a groove within a slot of the cap.

6. The apparatus of claim 1, wherein the tether is secured to the cap through a plurality of beads coupled to the tether wherein the beads frictionally secure the tether to the cap through a plurality of flaps extending across a slot within the cap.

7. The apparatus of claim 1, wherein the tether is secured to the cap through a plurality of knots coupled to the tether wherein the beads frictionally secure the tether to the cap through a plurality of flaps extending across a slot within the cap.

8. The apparatus of claim 1, wherein the cap and the tether comprise biodegradable polymers.

9. The apparatus of claim 1, wherein the cap sealingly engages the membrane of the joint capsule when coupled to the depot.

10. The apparatus of claim 1, wherein the depot is comprised of a biodegradable polymer wherein the biodegradable polymer is impregnated within the biological agent such that the biological agent contained within the depot is released over time.

11. The apparatus of claim 1, wherein the biological agent is selected from a group consisting of an analgesic, an anti-inflammatory, an antibiotic, an antiviral, a MMP inhibitor, and a growth factor.

12. The apparatus of claim 1, wherein the joint is a knee joint.

13. A method for providing a treatment to a joint comprising:
coupling a depot containing a biological agent to an interior surface of a membrane of a joint capsule wherein the depot degrades and releases the biological agent into the joint capsule.

14. The method of claim 13, wherein the depot is coupled to the interior surface of the membrane of the joint capsule through a tether and a cap, wherein the tether extends from the depot across the membrane of the joint capsule through at least one incision or hole in the membrane and secures the cap to the membrane on an exterior side of the joint capsule.

15. The method of claim 13, wherein the biological agent is selected from a group consisting of an analgesic, an antibiotic, an antiviral, a MMP inhibitor, and a growth factor.

16. The method of claim 13, wherein the joint capsule is a knee joint capsule.

17. The method of claim 16, wherein the joint capsule is affected with a trauma selected from a group consisting of trauma to the anterior cruciate ligament, posterior cruciate ligament, the medial collateral ligament, the lateral collateral ligament, the patellar ligament, the medial meniscus, the lateral meniscus and chondrol fractures.

18. The method of claim 13, wherein the joint is affected by a disorder selected from a group consisting of osteoarthritis, chondromalacia and rheumatoid arthritis.

19. An apparatus for providing prolonged treatment to a joint comprising:
a depot containing a biological agent;
a tether extending from the depot and extendable through an incision or hole in a membrane of a joint capsule; and
a cap wherein the tether couples the depot to the cap such that the depot is coupled to the membrane on an interior side of the joint capsule.

20. A method of implanting a depot into a joint comprising:
(a) making an incision or hole in a membrane of a joint capsule;
(b) inserting a depot into the joint capsule through the incision or hole in the membrane such that a tether extending from the depot passes through the incision or hole in the membrane to an exterior side of the joint capsule and the depot remains on an interior side of the joint capsule;
(d) coupling the tether to a cap on the exterior side of the joint capsule wherein coupling the cap to the tether secures the depot to the membrane of the joint capsule.

21. A method for providing prolonged treatment for osteoarthritis to a knee joint comprising:
(a) making an incision or hole in a membrane of a joint capsule of a knee joint;
(b) inserting a depot into the joint capsule through the incision or hole in the membrane wherein the depot contains a biological agent for treatment of osteoarthritis within the joint capsule such that a tether extends from the depot back through the incision or hole in the membrane and the depot remains on an interior side of the joint capsule;
(d) coupling the tether to a cap on the exterior side of the joint capsule wherein the tether couples the depot to the cap such that the depot is coupled to the membrane on an interior side of the joint capsule and the cap is coupled to the membrane on an exterior side of the joint capsule.
